(19) United States
(12) Patent Application Publication
(10) Pub. No.: US 2024/0082139 A1
(43) Pub. Date: Mar. 14, 2024

(54) USE IN AN AQUEOUS-ALCOHOLIC COMPOSITION OF POLYMERS OBTAINED BY LOW-CONCENTRATION INVERSE EMULSION POLYMERISATION WITH LOW NEUTRALISED MONOMER LEVEL

(71) Applicant: SNF GROUP, Andrezieux Boutheon (FR)

(72) Inventors: Frédéric BLONDEL, Andrezieux Boutheon (FR); Thomas BOURSIER, Andrezieux Boutheon (FR); Charlène BOTTE, Andrezieux Boutheon (FR)

(73) Assignee: SNF GROUP, Andrezieux Boutheon (FR)

(21) Appl. No.: 18/555,193

(22) PCT Filed: Apr. 19, 2022
(86) PCT No.: PCT/EP2022/060303

§ 371 Date: Oct. 12, 2023

(30) Foreign Application Priority Data
Apr. 22, 2021 (FR) FR2104192

(51) Int. Cl. A61K 8/81; A61K 8/04; A61K 8/34; A61Q 17/00

(52) U.S. Cl. CPC A61K 8/8147; A61K 8/042; A61K 8/34; A61Q 17/005; A61K 2800/48

(57) ABSTRACT

The present invention relates to the use, for the manufacture of a hydroalcoholic composition comprising 50% to 80% by weight of at least one alcohol and at least 10% by weight of an aqueous phase, of a branched or crosslinked polymer obtained by polymerization of an aqueous solution of one or more monomers in a water-in-oil inverse emulsion, at least one of the monomers used being an acrylic monomer and one or more of the monomers used being a monomer bearing at least one weak acid function, the molar percentage of monomers bearing at least one weak acid function relative to all the monomers used being at least 30%, and wherein: i) the polymerization is carried out with a concentration of all the monomers in aqueous solution belonging to the range from 1.3 mmol to 3.6 mmol per gram of aqueous solution, ii) during the polymerization, at most 20 mol % of the acid functions present on the monomers having at least one acid function are found in neutralized form.

The present invention relates to the field of hydroalcoholic compositions. More specifically, the invention relates to the use, for the manufacture of an hydroalcoholic composition, of a copolymer obtained by low-concentration inverse emulsion polymerization with a low rate of neutralized monomers.

### PRIOR STATE OF THE ART

Viruses cause some of the most common human illnesses, including the common cold, chicken pox and herpes. Some serious diseases, including those responsible for many epidemics in modern society, such as Ebola, AIDS, SARS and SARS-CoV-2, are also caused by viruses.

Viruses are infectious agents consisting of nucleic acid (DNA/RNA) in a protein coat that attaches to host cells of living organisms and replicates by forcing the host cell to copy the genetic material of the virus. As viruses can replicate in the body despite the host's defense mechanisms, they can cause lifelong chronic infections. In addition to their devastating ability to cause serious illnesses, viruses are also easily transmitted, being able to pass through bodily fluids, contaminated food/drink, insects and physical contact.

Compositions with a high alcohol content generally help to prevent the transmission of viruses and other pathogens through the skin because they allow virucidal efficacy without skin irritation. These hydroalcoholic compositions are skin sanitizing solutions and kill a wide range of viruses and other pathogens that may be present on the skin, especially on the hands. They require direct and mechanical contact (friction) and are used without water.

Thickening polymers are commonly added to hydroalcoholic compositions in order to give them rheological properties and, therefore, to make their application efficient and practical.

Different types of polymers formed from at least one monomer containing a weak acid function are generally used as thickening and/or stabilizing agent in different types of applications. For example, mention may be made of patents FR 2 810 545 and FR 2 873 126, or even U.S. Pat. No. 3,724,547.

Patent EP 1 047 716 describes polymers based on acrylic acid and 2-acrylamido-2-methylpropane sulfonic acid obtained by inverse emulsion polymerization. Although these polymers seem to have a thickening effect in an aqueous medium, they are not very effective in hydroalcoholic compositions.

Patent JPH 09157130 describes polymers based on acrylic acid and 2-acrylamido-2-methylpropane sulfonic acid obtained by different polymerization techniques. The exemplified polymers are obtained by dispersion polymerization.

However, the hydroalcoholic compositions known today are not satisfactory. Indeed, during their use, hydroalcoholic compositions tend to have a sticky feeling during application.

The problem which the applicant seeks to solve is to obtain hydroalcoholic compositions which exhibit minor sticky feeling during application and a greater feeling of softness, while having a satisfactory viscosity.

### DISCLOSURE OF THE INVENTION

Therefore, the purpose of the present invention is to propose the use, for the manufacture of a hydroalcoholic composition comprising 50% to 80% by weight of at least one alcohol and at least 10% by weight of an aqueous phase, of a branched or crosslinked polymer composed of the repetition of one or more monomeric units, in which at least one of the monomeric units comprises an acrylic group and at least 30 mol % of the monomeric units bear at least one weak acid function, optionally in neutralized form. Said polymer is obtained:
by polymerization of an aqueous solution of one or more monomers in a water-in-oil inverse emulsion, in which at least one of the monomers used is an acrylic monomer and at least one of the monomers used is a monomer bearing at least one weak acid function, the molar percentage of monomers bearing at least one weak acid function relative to all the monomers used being at least 30%, the aqueous phase of the aqueous solution containing at least one monomer acting as a branching agent, so that the polymerization leads to a branched or crosslinked polymer, and in which:
   i) the polymerization is carried out with a concentration of all the monomers in aqueous solution belonging to the range from 1.3 mmol to 3.6 mmol per gram of aqueous solution,
   ii) during the polymerization, at most 20 mol % of the acid functions present on the monomers having at least one acid function are in neutralized form.

The hydroalcoholic composition thus manufactured comprises 50% to 80% by weight of said at least one alcohol, at least 10% by weight of said aqueous phase, and the branched or crosslinked polymer as described above, the percentages being given with respect to the total weight of the composition.

The expression “during the polymerization” means that, at the start of the polymerization, at most 20 mol % of the acid functions present on the monomers having at least one acid function are in neutralized form. In other words, the neutralization is carried out prior to the polymerization.

The expression “with a concentration of all the monomers in aqueous solution” means that the sum of the concentrations by weight of all the monomers in aqueous solution belongs to the range from 1.3 mmol to 3.6 mmol per gram of aqueous solution. The total monomer concentration includes the monomers intended to form the monomer units of the branched or crosslinked polymer, known as “used” monomers, as well as the monomers acting as branching agents.

Polymerization is optionally followed by one or more of the following steps:
dilution or concentration of the emulsion obtained,
insulation to obtain the polymer in the form of a powder,
at least partial neutralization of the free acid functions present on the polymer obtained. Preferably, the neutralization is carried out so that the polymer comprises a percentage of neutralized acid functions of 30 to 100 mol % relative to all of the acid functions present on the polymer.

Such a polymer, defined by its process for obtaining it, is named “thickening polymer”, “acrylic polymer” or “branched or crosslinked polymer” in the following description.

The invention also relates to the use of such a thickening polymer, for thickening a hydroalcoholic composition in order to obtain a thickened hydroalcoholic composition comprising 50% to 80% by weight of at least one alcohol and at least 10% by weight of an aqueous phase. The thickened composition thus comprises 50% to 80% by weight of said at least one alcohol, at least 10% by weight of said aqueous phase, and said thickening polymer.

The invention also relates to a hydroalcoholic composition, comprising 50% to 80% by weight of at least one alcohol, at least 10% by weight of an aqueous phase, and a branched or crosslinked polymer consisting of the repetition of one or more monomeric units, in which at least one of the monomeric units, comprising an acrylic group and at least 30 mol % of the monomeric units, bears at least one weak acid function, at least partially in neutralized form.

Said polymer is obtained:
by polymerization of an aqueous solution of one or more monomers in a water-in-oil inverse emulsion, in which at least one of the monomers used is an acrylic monomer and at least one of the monomers used is a monomer bearing at least one weak acid function, the molar percentage of monomers bearing at least one weak acid function relative to all the monomers used being at least 30%, the aqueous phase of the aqueous solution containing at least one monomer acting as a branching agent, so that the polymerization leads to a branched or crosslinked polymer, and in which:
   i) the polymerization is carried out with a concentration of all the monomers in aqueous solution belonging to the range from 1.3 mmol to 3.6 mmol per gram of aqueous solution,
   ii) during the polymerization, at most 20 mol % of the acid functions present on the monomers having at least one acid function are in neutralized form.

The hydroalcoholic uses and compositions according to the invention preferably have one or the other of the characteristics below, or any combination of these characteristics, or even all of the characteristics below when they are not mutually exclusive:
during the polymerization, at most 10%, preferably at most 5%, and preferably at most 2%, of the acid functions present on the monomers having at least one acid function are in neutralized form; according to a particular embodiment, all the acid functions present on the monomers, i.e., 100% of the acid functions, are in the free acid form, during the polymerization; the percentages indicated are molar percentages;
the polymerization is carried out with a concentration of all the monomers in aqueous solution belonging to the range from 1.7 to 3.3 mmol per gram of aqueous solution;
the polymer comprises a molar percentage of monomeric units bearing one or more weak acid function(s), relative to all the monomeric units, of at least 50%, preferably of at least 70%, most preferably at least 80%;
all the monomers used for the preparation of the polymer are monomers having at least one ethylenic unsaturation;
the monomeric unit(s) bearing at least one weak acid function, in free form, is (are) chosen from acrylic acid, methacrylic acid, itaconic acid, crotonic acid, maleic acid and fumaric acid, acrylic acid being preferred;
the polymer is a copolymer comprising at least one neutral monomeric unit chosen from acrylamide, methacrylamide, N, N-dimethylacrylamide, N-vinylmethylacetamide, N-vinylformamide, vinyl acetate, diacetoneacrylamide, N-isopropyl acrylamide, N-[2-hydroxy-1,1-bis(hydroxymethypethyl]propenamide, (2-hydroxyethyl) acrylate, (2,3-dihydroxypropyl) acrylate, methyl methacrylate, (2-hydroxyethyl) methacrylate, (2,3-dihydroxy propyl) methacrylate and vinylpyrrolidone;
preferably:
   either all the monomeric units bearing at least one acid function present in the polymer are monomeric units bearing one or more weak acid function(s). In particular, the polymer present in the composition is an acrylic/acrylamide acid copolymer in which 30% to 100% of the acrylic acid functions are in neutralized form;
   or the polymer is a copolymer comprising at least one monomeric unit bearing one or more strong acid function(s). Preferably, the molar percentage of monomeric units bearing one or more strong acid function(s) relative to all of the monomeric units is less than 50%, and preferably less than 30%. Preferably, the monomeric unit(s) bearing one or more strong acid function(s), in free form, is (are) chosen from acrylamidoalkylsulfonic acids such as 2-acrylamido-2-methylpropane sulfonic acid. In particular, the polymer present in the composition is a 2-acrylamido-2-methylpropane sulphonic acid/acrylic acid or 2-acrylamido-2-methylpropane sulphonic acid/acrylic/acrylamide acid copolymer, in which 30% to 100% of the acid functions are in neutralized form;
the branching agent is chosen from methylenebisacrylamide (MBA), ethylene glycol di-acrylate, polyethylene glycol dimethacrylate, diacrylamide, cyanomethylacrylate, vinyloxyethylacrylate, vinyloxymethacrylate, triallylamine, tetraallylammonium chloride (TAAC), formaldehyde, glyoxal, glycidyl ethers such as ethylene glycol diglycidyl ether, epoxies, and mixtures thereof;
the amount of branching agent in the composition is between 5 ppm and 10,000 ppm by weight relative to the total weight of monomers, and preferably between 100 ppm and 5,000 ppm;
the polymerization reaction is carried out in the presence of a water-in-oil emulsifying agent;
the composition comprises from 0.01% to 10% by weight of branched or crosslinked polymer, relative to the total weight of the composition, and preferably from 0.1% to 5% by weight of branched or crosslinked polymer;
the composition comprises at least one additive, and in particular at least one formulation auxiliary, for example chosen from chelating agents, diluents, neutralizing agents and pH adjustment agents, opacifiers, preservatives, spreaders, emollients, film-forming polymers, antioxidants, fragrances, reflecting agents, coalescing agents and mixtures thereof;
the composition comprises from 60% to 70% by weight of at least one alcohol relative to the total weight of the composition;
the alcohol included in the hydroalcoholic composition is advantageously chosen from methanol, ethanol, isopropanol, butanol, pentanol, hexanol, their isomers and mixtures thereof. Preferably, the alcohol is chosen from ethanol and/or isopropanol;
the hydroalcoholic composition comprises:
   a) 50% to 80% by weight of said at least one alcohol,
   b) 0.1% to 10% by weight of the branched or crosslinked copolymer,
   c) 10% to 49.9% by weight of water,
   d) 0% to 10% by weight of at least one additive.
the hydroalcoholic composition comprises:
   a) 60% to 75% by weight of at least one alcohol,
   b) 0.1% to 5% by weight of the branched or crosslinked copolymer,
   c) 25% to 39.9% by weight of water,
   d) 0% to 10% by weight of at least one additive.

The polymers used in the context of the invention and their method of production will first be described.

The polymers used in the context of the invention consist of the repetition of one or more monomeric units, with at least one of the monomeric units, which corresponds to a monomer, containing an acrylic group. In other words, these polymers correspond to homopolymers obtained by polymerization of a monomer comprising an acrylic group, or to copolymers obtained by copolymerization of a mixture of monomers, at least one of which comprises an acrylic group. For the sake of simplicity, in the rest of the description, such polymers could simply be called “acrylic polymers”.

In order to effectively fulfill their thickening role, the polymers used in the context of the invention are water-soluble or water-swellable. The monomers used for the preparation of these polymers and, in particular, the rate of hydrophilic monomers, will be selected so as to obtain such properties.

The expression “water-soluble polymer” is understood to mean a polymer which, when dissolved by stirring in water at a temperature of 25° C. at a concentration of 50 g/I, gives a solution free of insoluble particles.

The expression “water-swellable polymer” is understood to mean a polymer which, when dissolved in water at a temperature of 25° C., swells and thickens the solution.

The polymers used in the context of the invention are branched or crosslinked. The expression “branched polymers” is understood to mean, conventionally, non-linear polymers which have side chains. Branched polymers include, in particular, star-shaped and comb-shaped polymers. The expression “crosslinked polymer” is understood to mean, conventionally, a non-linear polymer which is in the form of a three-dimensional network insoluble in water, but swellable in water.

The crosslinking is obtained by implementing a branching agent during the polymerization which is integrated into the aqueous phase. Such a branching agent corresponds to a monomer comprising two or more ethylenic unsaturations. It is, for example, chosen from methylenebisacrylamide (MBA), ethylene glycol diacrylate, polyethylene glycol dimethacrylate, diacrylamide, cyanomethylacrylate, vinyloxyethylacrylate, vinyloxymethacrylate, triallylamine, tetraallylammonium chloride (TAAC), formaldehyde, glyoxal, glycidyl ethers such as ethylene glycol diglycidyl ether, epoxies and mixtures thereof.

It should be specified that, in the context of the invention, the total concentration of monomers given in relation to the polymerization process includes the monomers acting as branching agents.

In the context of the invention, the applicant was interested in using acrylic polymers corresponding to, or obtained from, an inverse emulsion of polymers prepared by water-in-oil inverse emulsion polymerization with the use of a high molar percentage of monomers bearing one or more weak acid function(s) relative to all of the monomers used, and, in particular, comprising at least 30 mol % of monomers bearing at least one weak acid function. With such a rate of monomers bearing a weak acid function, the inventors have demonstrated that the properties of the polymer obtained are really dependent, on the one hand, on the rate of neutralization of the acid functions of the monomers used during the polymerization and, on the other hand, of the total concentration of monomers in the aqueous phase. In a novel manner compared to the approaches proposed in the prior art, which recommend carrying out the polymerization with a high rate of neutralization of the acid functions, the applicant chose, within the scope of the invention, an inverse emulsion polymerization process of polymers having a low rate of neutralization and, in particular, a rate of neutralization of the acid functions of at most 20 mol %.

In the context of the invention, the applicant proposes to use such a polymer, obtained by polymerization of an aqueous solution of monomers in water-in-oil inverse emulsion, in which the polymerization is carried out with a concentration of all the monomers belonging to the range from 1.3 mmol to 3.6 mmol per gram of aqueous solution. In addition, the applicant demonstrated that such a range of concentrations, unlike the higher concentrations used, in particular, in the prior art, was compatible with obtaining a polymer with a low rate of neutralization of the weak acid functions present and made it possible to overcome the stability problems noted in the prior art.

In the context of the invention, the polymer used is obtained by implementing a process for preparing a polymer by polymerization of an aqueous solution of one or more monomers in a water-in-oil inverse emulsion, in which one or more of the monomers used comprise at least one acid function, the molar percentage of monomers bearing at least one weak acid function relative to all the monomers used being at least 30%, and in which:
i) the polymerization is carried out with a concentration of all the monomers in aqueous solution belonging to the range from 1.3 mmol to 3.6 mmol per gram of aqueous solution,
ii) during the polymerization, at most 20 mol % of the acid functions present on the monomers used having at least one acid function are in neutralized form.

In particular, during the polymerization, at most 10%, preferably at most 5%, and preferably at most 2%, of the acid functions present on the monomers used having at least one acid function are in neutralized form, which allows to obtain even more advantageous thickening properties. According to a particular embodiment, 100% of the acid functions present on the monomers used are in the free acid form, during the polymerization.

In the context of the invention, in an optimal manner, the polymerization is carried out with a total concentration of monomers present in the aqueous solution belonging to the range from 1.7 to 3.3 mmol per gram of aqueous solution. In the context of the invention, the monomer concentrations are given relative to the total weight of aqueous solution (also called aqueous phase), i.e., weight of monomers included.

In particular, it is therefore possible to carry out the polymerization with the following combinations:
A concentration of all the monomers in aqueous solution belonging to the range from 1.3 mmol to 3.6 mmol per gram of aqueous solution, with at most 20 mol %, advantageously at most 10%, preferably at most 5%, and preferably at most 2%, or even 0% of the acid functions present on the monomers having at least one acid function which are in neutralized form,
A concentration of all the monomers in aqueous solution belonging to the range from 1.7 mmol to 3.3 mmol per gram of aqueous solution, with at most 20 mol %, advantageously at most 10%, preferably at most 5%, and preferably at most 2%, or even 0% of the acid functions present on the monomers having at least one acid function which are in neutralized form.

The molar percentage of monomers bearing at least one weak acid function relative to all the monomers used is preferably at least 50%, preferably at least 70%, most preferably at least 80%. Such molar percentages may be used with any of the monomer concentration/neutralization rate combinations mentioned above.

In the context of the invention, the polymerization will preferably be carried out with monomers which all have at least one ethylenic unsaturation.

Preferably, the polymerization is carried out with a single monomer bearing at least one weak acid function, the molar percentage of which relative to all the monomers used is at least 30%, which, in free form, is chosen from acrylic acid, methacrylic acid, itaconic acid, crotonic acid, maleic acid and fumaric acid. The monomer bearing at least one weak acid function is most preferably acrylic acid in free form, or with a rate of neutralization in accordance with the invention. It is also possible to use several monomers bearing at least one weak acid function, in particular chosen from those listed above, the total molar percentage of which relative to all the monomers used is at least 30%. Preferably, one of these monomers is acrylic acid in free form, or with a rate of neutralization in accordance with the invention.

The polymerization may be carried out with at least one monomer bearing at least one strong acid function. In this case, the polymerization is preferably carried out with a concentration of monomers bearing at least one strong acid function relative to all the monomers used of less than 50%, and preferably less than 30%. The polymerization may, for example, be carried out with a monomer bearing at least one strong acid function which, in free form, is chosen from acrylamidoalkylsulfonic acids, such as 2-acrylamido-2-methylpropane sulfonic acid (AMPS). In this case, the polymerization may, for example, be carried out with an acrylic acid/AMPS or acrylic acid/AMPS/acrylamide combination, the acid monomers possibly being in free form or with a rate of neutralization in accordance with the invention.

In the context of the invention, it was found that by selecting a concentration of monomers belonging to the range from 1.3 mmol to 3.6 mmol per gram of aqueous solution to carry out the polymerization reaction in inverse emulsion, it was possible to prepare inverse emulsions of polymers bearing an acid function with a low rate of neutralization, or even no neutralization, which are stable, i.e., without observing a rapid phenomenon of precipitation. In addition, it was demonstrated that such a range of concentrations, unlike the higher concentrations used, in particular, in the prior art, associated with a low neutralization of the acid functions present, made it possible to obtain polymers providing better sensory properties when applied, after an at least partial neutralization step, superior to the polymers of the prior art obtained by inverse emulsion polymerization.

The expression “monomer bearing at least one acid function” is understood to mean a monomer carrying one or more free or neutralized acid function(s) (i.e., salified by the action of a base). Therefore, the expression “acid function”, without further precision, designates both the acid functions in the free form and the neutralized form. When a monomer has more than one acid function, it is possible to have only part of the acid functions in neutralized form. The acid function(s) present may be a weak acid function or a strong acid function. In general, the monomers used will contain only weak acid functions or only strong acid functions, and, most often, monomers bearing a single acid function will be used. The same definitions and preferences apply to the monomeric units present on the polymer obtained.

By way of example of a monomer bearing at least one weak acid function in free form, of the —COOH type, mention may be made of acrylic acid, methacrylic acid, itaconic acid, crotonic acid, which all have a single weak acid function, and maleic acid and fumaric acid which have two weak acid functions.

As an example of monomer bearing a strong acid function in free form, mention may be made of monomers bearing a phosphonic acid or sulphonic acid function such as acrylamidoalkyl sulphonic acids such as 2-acrylamido-2-methylpropane sulfonic acid.

In their neutralized form, the acid functions are in anionic form with a counterion or cation depending on the base used for neutralization, for example of the Na⁺ type when soda is used, or NH₄⁺ when ammonia is used. Conventionally, the control of the number of acid functions in neutralized form is ensured by the choice of the pH of the aqueous solution of monomers which will be adjusted according to the pKa of the acid functions present.

The polymerization may involve a single type of monomer, then chosen from monomers bearing at least one weak acid function, or different types of monomers, of which at least one bears at least one weak acid function, with a proportion of acid functions present on the monomers used, and therefore on the copolymer obtained, in a neutralized form which is less than or equal to 20 mol %. In particular, in addition to the monomer unit(s) previously described which bear at least one weak acid function, the polymer obtained may contain other monomer units such as monomer units bearing at least one strong acid function, neutral (or non-ionic) monomer units, cationic monomeric units and/or monomeric units of a hydrophobic character. Whatever the case, the conditions of formation of the aqueous phase and polymerization are such that the acid functions of the monomers involved remain mainly in free form, and are not neutralized by formation of a salified form, or weakly neutralized with a limited neutralization rate less than or equal to 20 mol %. When a neutralization less than or equal to 20 mol % takes place, it is generally carried out in the aqueous phase, by adding an appropriate amount of base. A base such as soda or ammonia may be used.

In particular, the polymerization reaction may be carried out with at least one neutral monomer chosen from acrylamide, methacrylamide, N,N-dimethylacrylamide, N-vinylmethylacetamide, N-vinylformamide, vinyl acetate, diacetoneacrylamide, N-isopropyl acrylamide, N-[2-hydroxy-1,1-bis(hydroxymethyl)ethyl]propenamide, (2-hydroxyethyl) acrylate, (2,3-dihydroxypropyl) acrylate, methacrylate methyl methacrylate, (2-hydroxyethyl) methacrylate, (2,3-dihydroxy propyl) methacrylate, vinylpyrrolidone, or other acrylic esters, or other ethylenically unsaturated esters. For example, the polymerization may be carried out with from 30 to 99 mol % of at least one monomer having one or more weak acid function(s) and from 1 to 70 mol % of at least one neutral monomer. The polymerization may, for example, be carried out with an acrylic acid/acrylamide combination, the acrylic acid being in neutral form or with a rate of neutralization in accordance with the invention.

It is also possible to carry out a copolymerization with at least one cationic monomer. By way of example of cationic monomers, mention may be made of diallyldialkyl ammonium salts such as diallyldimethylammonium chloride (DADMAC); acidified or quaternized salts of dialkylaminoalkyl acrylates and methacrylates, in particular dialkylaminoethyl acrylate (ADAME) and dialkylaminoethyl methacrylate (MADAME); acidified or quaternized salts of dialkylaminoalkylacrylamides or methacrylamides, such as, for example, methacrylamido-propyl trimethyl ammonium chloride (MAPTAC), acrylamido-propyl trimethyl ammonium chloride (APTAC) and Mannich products such as quaternized dialkylaminomethylacrylamides.

The acidified salts are obtained by means known to a person skilled in the art, and, in particular, by protonation. Quaternized salts are also obtained by means known to a person skilled in the art, in particular by reaction with benzyl chloride, methyl chloride (MeCl), aryl or alkyl chlorides, or dimethyl sulfate.

It is also possible to carry out a copolymerization with at least one hydrophobic monomer. By way of example of hydrophobic monomers, mention may be made of acrylamidoundecanoic acid, undodecyl acid methyl acrylamide, acrylic acid derivatives such as alkyl acrylates or methacrylates such as, for example, ethoxylated behenyl methacrylate. In this case, the molar percentage of hydrophobic monomers relative to all the monomers used is less than 10%, and generally between 0.001% and 7%.

According to a first variant of the process according to the invention, all the monomers bearing at least one acid function used to carry out the polymerization are monomers bearing at least one weak acid function.

According to a second variant of the process according to the invention, the polymerization is carried out with at least one monomer bearing at least one strong acid function, in addition to at least one monomer bearing at least one weak acid function. In this case, the molar percentage of monomers bearing at least one strong acid function relative to all the monomers used is preferably less than 50%, most preferably less than 30%.

The copolymers obtained according to the process of the invention may, in particular, be formed from a combination of at least one monomer unit bearing at least one weak acid function and at least one monomer unit bearing at least one strong acid function, and, in particular, correspond to an acrylic acid/AMPS copolymer, these acid monomers being in neutral form or with a rate of neutralization in accordance with the invention; a combination of at least one monomeric unit bearing at least one weak acid function with at least one neutral monomeric unit and, optionally, at least one monomeric unit bearing at least one strong acid function, and, in particular, corresponding to an acrylic acid/acrylamide copolymer or to an acrylic acid/AMPS/acrylamide copolymer, the acrylic acid and the AMPS being in neutral form or with a rate of neutralization in accordance with the invention; a combination of at least one monomeric unit bearing at least one weak acid function with at least one cationic monomeric unit and, optionally, at least one monomeric unit bearing at least one strong acid function; or a combination of at least one monomer unit bearing at least one weak acid function with at least one neutral monomer unit and at least one cationic monomer and, optionally, at least one monomer unit bearing at least one strong acid function.

In the inverse emulsion polymerization process used in the context of the invention, the monomers are placed in aqueous solution. This aqueous solution corresponds to the aqueous phase of the inverse emulsion. In the context of the invention, in the aqueous solution used for the polymerization, at most 20 mol % of the acid functions present on the monomers having at least one acid function are in neutralized form.

It is possible to use a transfer agent, otherwise known as a “chain limiting agent”. The use of a transfer agent is particularly advantageous for controlling the molecular weight of the polymer obtained. By way of example of transfer agent, mention may be made of methanol, isopropanol, sodium hypophosphite, 2-mercaptoethanol, sodium methallysulfonate and mixtures thereof. In a known manner, a person skilled in the art will adjust the amounts of branching agent used and, optionally, of transfer agent, depending on whether they wish to obtain a branched or crosslinked polymer.

In more detail, the method used in the context of the invention comprises the following steps:
a) having an aqueous solution of the selected monomer(s), called the aqueous phase,
b) emulsifying said aqueous solution in a water-immiscible phase, called the oil phase,
c) carrying out the polymerization reaction.

Naturally, the aqueous solution of step a) has a total concentration of monomers, a molar percentage of monomers bearing at least one weak acid function relative to all the monomers used and a rate of neutralization of the acid functions present on the monomers having at least one acid function in accordance with the process described within the scope of the invention.

In general, the polymerization reaction is carried out in the presence of a water-in-oil emulsifier. The latter is most often introduced into the oil phase in which the aqueous solution is emulsified. The expression “emulsifying agent of the water-in-oil (W/O) type” is understood to mean an emulsifying agent having an HLB value low enough to provide water-in-oil emulsions, and, in particular, an HLB value of less than 10.

The HLB value is calculated according to the following relationship:

HLB=(% by weight of the hydrophilic part)/5

the percentage by weight of the hydrophilic part being the ratio between the molecular weight of the hydrophilic part and the total molecular weight of the molecule.

By way of example of such water-in-oil emulsifiers, mention may be made of surfactant polymers such as polyesters with a molecular weight between 1000 and 3000, products of the condensation between a poly(isobutenyl) succinic acid or its anhydride and a polyethylene glycol, block copolymers with a molecular weight between 2500 and 3500, for example those marketed under the names HYPERMER®, sorbitan extracts, such as sorbitan monooleate, sorbitan isostearate or sorbitan sesquioleate, certain esters of polyethoxylated sorbitans, such as pentaethoxylated sorbitan monooleate or pentaethoxylated sorbitan isostearate, or diethoxylated oleocetyl alcohol and tetraethoxylated lauryl acrylate.

In the inverse emulsion polymerization process, the aqueous solution contains the monomer(s) and, optionally, the branching agent and the transfer agent. It may also contain complexing agents such as ethylene diamine or ethylene diamine tetraacetic acid. Most often, the polymerization reaction of step c) is initiated by introducing into the emulsion formed in step b) a free radical initiator. By way of example of free radical initiators, mention may be made of oxidant-reducer couples with, among the oxidants, cumene hydroperoxide or tertiary butylhydroxyperoxide, and, among the reducers, persulphates such as sodium metabisulphite and Mohr's salt. Azo compounds such as 2,2′-azobis(isobutyronitrile) and 2,2′-azobis(2amidinopropane) hydrochloride may also be used.

Conventionally, the polymerization is generally carried out in an isothermal, adiabatic or temperature-controlled manner. That is to say that the temperature is maintained constant, generally between 10 and 50° C. (isothermal), or else the temperature is allowed to increase naturally (adiabatic) and, in this case, the reaction is generally started at a temperature below 10° C. and the final temperature is generally greater than 50° C., or, lastly, the rise in temperature is controlled to obtain a temperature curve between that of the isothermal and that of the adiabatic temperatures.

It is possible to introduce, at the end of the polymerization reaction, one or more oil-in-water emulsifying agents, preferably at a temperature below 50° C.

The expression “emulsifying agent of the oil-in-water (O/W) type” is understood to mean an emulsifying agent possessing a sufficiently high HLB value to provide oil-in-water emulsions and, in particular, an HLB value greater than 10. By way of example of such oil-in-water emulsifying agents, mention may be made of ethoxylated sorbitan esters such as ethoxylated sorbitan oleate with 20 equivalents of ethylene oxide (EO 20, i.e. 20 repetitions of the ethylene oxide unit in the molecule), laurate polyethoxylated sorbitan with 20 moles of ethylene oxide, polyethoxylated castor oil with 40 moles of ethylene oxide, decaethoxylated oleodecyl alcohol, heptaethoxylated lauryl alcohol, or polyethoxylated sorbitan monostearate with 20 moles of ethylene oxide.

The amounts of emulsifying agent(s) introduced are such that the inverse emulsion obtained will generally contain from 1% to 10% by weight, and preferably from 2.5% to 9% by weight, of emulsifying agents of the water-in-oil (W/O) type and, optionally, from 2% to 10% by weight, and preferably from 2.5% to 6% by weight of emulsifying agents of the oil-in-water (O/W) type.

In general, the weight ratio of the aqueous phase to the oil phase is from 50/50 to 90/10.

The oil phase used in the inverse emulsion polymerization process may be composed, for example, of a mineral oil, in particular commercial oil, containing saturated hydrocarbons of the paraffinic, isoparaffinic, cycloparaffinic, naphthalic type having a density between 0.7 and 0.9 at room temperature (22° C.); a vegetable oil; a synthetic oil, such as hydrogenated polydecene or hydrogenated polyisobutene; an ester, such as octyl stearate or butyl oleate; a vegetable oil, such as squalane or hemisqualane; or a mixture of several of these oils.

At the end of the polymerization reaction, it is also possible to dilute or concentrate the emulsion obtained. In particular, it is possible to concentrate the emulsion obtained by distillation, or to completely dry it in order to obtain a powder. Such concentration or drying will be carried out with or without the prior introduction of an emulsifying agent of the oil-in-water (O/W) type.

The inverse emulsions thus obtained may be concentrated, for example by distillation. Inverse emulsions are then obtained, the polymer concentration of which may be between 30 and 75% by weight, preferably between 40 and 65% by weight.

The polymers obtained from the inverse emulsions subsequently subjected to an isolation step may be found in the form of a powder. Such an isolation step may, for example, be chosen from the techniques of precipitation, azeotropic distillation and drying by atomization and spraying.

Indeed, in the context of the invention, it is possible to concentrate or isolate the polymer in the form of an inverse emulsion obtained directly at the end of the inverse emulsion polymerization process, without losing the advantageous properties of the polymers obtained. There are, in particular, many processes for obtaining powder from inverse emulsions of polymers which consist in isolating the active ingredient from the other constituents of the emulsion, such as, for example:
precipitation in a non-solvent medium such as acetone, methanol or any other polar solvent in which the polymer is not soluble. A simple filtration then makes it possible to isolate the polymer particle.
azeotropic distillation in the presence of an agglomerating agent and a stabilizing polymer makes it possible to produce agglomerates which are easily isolated by filtration before drying the particle.
“spray-drying” which consists in creating a cloud of fine droplets of emulsion in a stream of hot air for a controlled period.

The polymers obtained after such steps keep their advantageous properties, in terms of thickening capacity and resistance to electrolytes.

Without an additional neutralization step, at the end of the inverse emulsion polymerization process, or after a drying or concentration step, at most 20 mol % of the acid functions present on the polymers obtained are in neutralized form, preferably at most 10%, even more preferably at most 5%, and preferably at most 2%. This low rate of neutralization of the acid functions present offers the formulator great flexibility, allowing them to adjust the properties of the polymer and, therefore, the desired thickening effect, by adjusting the rate of neutralization as required. Such an approach also allows the formulator to choose the nature of the neutralizing agent used, compatible with the targeted use.

In order to obtain the desired effects, the polymerization is usually followed by a neutralization step, otherwise called a “post-neutralization step”, of at least some, or even all, of the free acid functions present on the polymer. In the case where a step of at least partial neutralization of the free acid functions present on the polymer obtained is carried out after the polymerization reaction, it preferably leads to a percentage of neutralization from 30 to 100% with respect to all of the acid functions present on the polymer.

Such a post-neutralization step may be carried out in different ways:
post-neutralization may be done on the inverse emulsion obtained at the end of the inverse emulsion polymerization process. Generally, this is the case when the manufacturer neutralizes the polymer himself in the form of an inverse emulsion.
post-neutralization may be done on an aqueous solution obtained following the inversion of the inverse emulsion in water. Generally, this is the case when the formulator implements the inverse emulsion, or the resulting powder, in an aqueous solution, called “stock solution”, before adding the latter to the composition to be thickened. He then has the freedom to adjust the polymer concentration of the solution, the neutralization rate and the nature of the neutralizing agents.
post-neutralization may also be carried out on the composition in which the inverse emulsion or the resulting powder was incorporated. In the same way as the previous case, the user has the freedom to adjust the neutralization rate and the nature of the neutralizing agents.

The neutralization is carried out using a base, in a manner similar to the neutralization of the monomers previously described in the context of the polymerization process, the nature and amounts of which are selected by a person skilled in the art.

These polymers thus neutralized offer much better thickening properties and better sensory properties, all other conditions being equal, compared to the polymers obtained by inverse emulsion polymerization, which do not satisfy the conditions of concentration and neutralization of the monomers as defined in the process according to the invention. Particularly after neutralization, the polymers offer advantageous properties over polymers made from the same monomers, but prepared by reverse emulsion polymerization directly at higher neutralization rates and/or at a different total monomer concentration.

Advantageously, the polymers used in the context of the invention make it possible, after complete neutralization of the free acid functions present, or at least greater neutralization, to much more effectively thicken the aqueous media present in hydroalcoholic compositions.

The process for preparing the hydroalcoholic compositions according to the invention, and, in particular, the incorporation of the polymers previously described, will now be described in detail.

The manufacture of hydroalcoholic compositions is widely known to a person skilled in the art. The addition of the thickening acrylic polymer described above may be done at any stage of the manufacture of the hydroalcoholic composition. Preferably, in a first step, an aqueous solution which may contain additives is thickened by means of the thickening agent. In a second step, the alcohol or alcohols are gradually added under stirring. If neutralization is necessary, it is preferably carried out at the end of the preparation, in the case of thickening polymer, in the form of an inverse emulsion.

The hydroalcoholic composition preferably comprises from 0.01% to 10% by weight of a thickening acrylic polymer, and preferably from 0.1 to 5% by weight, these percentages being given relative to the total weight of the hydroalcoholic composition.

The hydroalcoholic composition has a viscosity of between 500 and 8000 cps, preferably between 700 and 6000 cps, more preferably between 1000 and 5500 cps. The viscosity is measured at 25° C. with a Brookfield RVT viscometer (rotational speed of 20 rpm), belonging to the range from 2,000 mPa·s to 100,000 mPa·s, in particular to the range from 3,000 mPa·s to 50,000 mPa·s.

The neutralization step leading to a percentage of neutralized acid functions of 30 to 100% relative to all the acid functions present on the polymer may be done before or after the incorporation of the polymer into the composition.

Furthermore, the polymer obtained by inverse emulsion polymerization, according to the process described above, retains its advantageous properties, whether it is in the form of a more or less concentrated inverse emulsion, a powder or an aqueous solution. Consequently, the thickening acrylic polymer may be used in the form of an inverse emulsion, a powder or in solubilized form, for example in water, or else in the form of an aqueous or organic dispersion. Generally, a solubilized form of the polymer in water is used, obtained either by inversion of an inverse emulsion in water, or by dissolving a powder in water. At the time of its use, whatever the form in which it is introduced, the polymer plays its role of thickener.

Conventionally, the hydroalcoholic compositions according to the invention may comprise at least one additive, the content of which is from 0 to 10% by weight of the composition. The additive is advantageously chosen from surfactants, pH adjusting agents, perfumes, dyes, preservatives, and moisturizing agents, antibacterial agents, or anti-complexing agents. Such active and additive agents are well known to the formulator of hydroalcoholic compositions, in particular in the fields of cosmetics and dermatology.

Preferably, the hydroalcoholic composition comprises at least one moisturizing agent. The moisturizing agent may be chosen from allantoin; pyrrolidonecarboxylic acid and salts thereof; hyaluronic acid and salts thereof; sorbic acid and salts thereof; amino acids, for example urea, lysine, arginine, cysteine, or guanidine; polyhydroxy alcohols such as glycerin, propylene glycol, hexylene glycol, hexanetriol, ethoxydiglycol, dimethiconecopolyol and sorbitol, as well as the esters thereof; polyethylene glycol; glycolic acid and glycolate salts (e.g. ammonium and quaternary alkyl ammonium); chitosan; aloe vera extracts; seaweed extracts; honey and extracts thereof; inositol; lactic acid and lactate salts (e.g. ammonium and quaternary alkyl ammonium); D-panthenol; magnesium ascorbyl phosphate; kojic acid; lactamide-monoethandamine; acetamide-monoethanolamine; their analogs and mixtures thereof. Preferably, the moisturizing agent is glycerin.

Preferably, the hydroalcoholic composition comprises:
a) 50% to 80% by weight of at least one alcohol,
b) 0.1% to 10% by weight of at least one branched or crosslinked copolymer as described above,
c) 10% to 49.9% by weight of water,
d) 0% to 10% by weight of at least one additive.

More preferably, the hydroalcoholic composition comprises:
a) 60% to 75% by weight of at least one alcohol,
b) 0.1% to 5% by weight of at least one branched or crosslinked copolymer as described above,
c) 25% to 39.9% by weight of water,
d) 0% to 10% by weight of at least one additive.

Preferably, the hydroalcoholic composition comprises between 0.1% and 10% by weight of at least one moisturizer, preferably from 0.5% to 8% by weight, and more preferably, from 1% to 5% by weight, relative to the total weight of the composition.

The invention and the resulting advantages will emerge better from the following examples

### DESCRIPTION OF THE FIGURE

The FIGURE below illustrates in a non-limiting manner the advantages and characteristics of the invention:

FIG. 1 is a graphic representation of the results of the sensory analyzes of the hydroalcoholic compositions.

### EXAMPLES OF EMBODIMENTS OF THE INVENTION

### Example 1 (According to the Invention)

The ingredients of the aqueous phase are loaded into a 1 L beaker under magnetic stirring:
150 g of glacial acrylic acid
600 g of deionized water
0.023 g of sodium hypophosphite (150 ppm)
0.10 g of sodium diethylenetriaminepentacetate
0.075 g of methylenebisacrylamide (500 ppm)
0.15 g of sodium bromate
5.8 g of sodium hydroxide

Then, in a 1 L glass reactor, under mechanical stirring, the organic phase is prepared with:
102 g of aliphatic hydrocarbon (Isopar L)
98 g of white mineral oil (Marcol 152)
20 g of sorbitol monooleate
25 g of polymeric stabilizer (Hypermer 1083).

The aqueous phase is gradually transferred into the organic phase. The pre-emulsion thus formed is then subjected to high shear for 1 minute (Ultra Turrax, IKA).

The inverse emulsion is then degassed for 30 min by simple nitrogen sparging.

An aqueous solution containing 1.0% by weight of sodium metabisulphite is then added at a rate of 2.5 ml/h for a period of 1.5 hours. Once the maximum temperature is reached, the temperature of the reaction mixture is maintained for 60 min before cooling.

Finally, 40 g of ethoxylated tridecyl alcohol (6 moles) are added when the temperature is around 30° C.

### Example 2 (According to the Invention)

The ingredients of the aqueous phase are loaded into a 1 L beaker under magnetic stirring:
150 g of glacial acrylic acid
605 g of deionized water
0.10 g of sodium diethylenetriaminepentacetate
0.225 g of methylenebisacrylamide (1500 ppm)
0.15 g of sodium bromate

Then, in a 1 L glass reactor, under mechanical stirring, the organic phase is prepared with:
102 g of aliphatic hydrocarbon (Isopar L)
98 g of white mineral oil (Marcol 152)
20 g of sorbitol monooleate
25 g of polymeric stabilizer (Hypermer 1083).

The aqueous phase is gradually transferred into the organic phase. The pre-emulsion thus formed is then subjected to high shear for 1 minute (Ultra Turrax, IKA).

The inverse emulsion is then degassed for 30 min by simple nitrogen sparging.

An aqueous solution containing 1.0% by weight of sodium metabisulphite is then added at a rate of 2.5 ml/h for a period of 1.5 hours. Once the maximum temperature is reached, the temperature of the reaction mixture is maintained for 60 min before cooling.

Finally, 40 g of ethoxylated tridecyl alcohol (6 moles) are added when the temperature is around 30° C.

Other examples 3 to 6, in accordance with the invention, are prepared according to the protocol of Example 1, by varying the molar percentage of weak acid with respect to all the monomers, as well as the molar percentage of neutralized weak acid.

Counter-examples 1 to 4, not in accordance with the invention, are prepared according to the protocol of Example 1, by varying the concentration of all the monomers in aqueous solution, the molar percentage of weak acid with respect to all the monomers, as well as the molar percentage of neutralized weak acid.

Preparation of the Hydroalcoholic Composition

82.5 g of deionized water are added to a beaker, then the amount of inverse emulsion (Examples 1 to 6) desired is gradually added under mechanical stirring (three-bladed—500 rpm) to obtain a solution containing the percentage by weight of desired active polymer. The solution is stirred for 5 min to invert the emulsion. Then, 4.5 g of glycerin are added to the solution. After stirring for 1 min, the ethanol is gradually introduced and stirred until the medium is completely homogenized.

The viscosity is then measured using a Brookfield RVT viscometer with module 4 and 20 rpm speed of rotation.

The polymers according to the invention are compared with a commercially available thickening polymer. It is the SEPIGEL® 305 (marketed by SEPPIC) which is an inverse emulsion. This commercial product is typically used in hydroalcoholic compositions as a thickening agent.

TABLE
Composition of the hydroalcoholic composition

| Product | Weight % |
|---|---|
| Water | QSP |
| Polymer | See Tables 2 and 3 |
| Glycerine | 1.5 |
| Ethanol | 70 |

TABLE 2
Characteristics of hydroalcoholic compositions

| | | Aqueous | | Neu- | | GHA |
|---|---|---|---|---|---|---|
| | Aqueous | phase | Weak | tralized | | Viscosity |
| | phase | [mono- | acid | weak | | 0.5% |
| | weight | mers] | mol | acid | Proces- | polymer |
| | (g) | (mmol/g) | % | mol % | sability | (cps) |
| Ex 1 | 756.15 | 2.76 | 100 | 5 | OK | 3800 |
| Ex 2 | 755.5 | 2.76 | 100 | 0 | OK | 1900 |
| Ex 3 | — | 2.76 | 70 | 0 | OK | 1300 |
| Ex 4 | — | 2.76 | 50 | 0 | OK | 950 |
| Ex 5 | — | 2.76 | 30 | 0 | OK | 650 |
| Ex 6 | — | 2.76 | 100 | 19 | OK | 1200 |
| SEPIGEL ® | — | — | 0 | — | — | 620 |
| 305 | | | | | | |
| CEx 1 | — | 2.76 | 100 | 25 | OK | Precip- |
| | | | | | | itation |
| CEx 2 | — | 4 | 100 | 5 | Un- | NA |
| | | | | | stable | |
| CEx 3 | — | 2.76 | 20 | 0 | OK | <100 |
| CEx 4 | — | 1 | 100 | 0 | OK | <100 |

In Table 2, “Aqueous phase weight” corresponds to the weight of the aqueous phase in the composition, in grams, and “Aqueous phase [monomers]” corresponds to the concentration of all the monomers in aqueous solution, in mmol per gram of aqueous solution.

Table 3 below details the viscosities obtained for examples 1 and 2 as well as SEPIGEL® 305, for different weight percentages of polymer.

TABLE 3
Measurement of the viscosity of hydroalcoholic compositions

| | Polymer | Viscosity |
|---|---|---|
| Product | weight % | (cps) |
| SEPIGEL ® | 0.80% | 1950 |
| 305 | 1.00% | 3500 |
| Example 1 | 0.20% | 510 |
| | 0.30% | 2100 |
| | 0.50% | 3800 |
| | 0.80% | 5800 |
| | 1.00% | 6000 |
| Example 2 | 0.30% | 100 |
| | 0.50% | 1900 |
| | 1.00% | 4500 |

To qualify as a gel, a composition must have a viscosity greater than 500 cps.

The polymers of Examples 1 to 6 give more viscous compositions than SEPIGEL® 305 at identical concentration. Their viscosity being greater than 500 cps, these compositions are gels.

With regard to the counter-examples, it should be noted that a percentage of neutralized weak acid greater than 20% (CEx 1), all other parameters remaining unchanged, leads to the precipitation of the polymer, and thus to an unsatisfactory hydroalcoholic composition.

A concentration of all the monomers in aqueous solution greater than 3.6 mmol per gram of aqueous solution (CEx 2), or less than 1.3 mmol per gram of aqueous solution (CEx 4), any other parameter remaining unchanged, leads respectively to an unstable composition whose viscosity cannot be measured, or to a composition whose viscosity is lower than that of SEPIGEL® 305, and, in particular, lower than 100 cps. These compositions are not gels.

A molar percentage of monomers bearing at least one weak acid function relative to all the monomers used of less than 30 mol % (CEx 3), any other parameter remaining unchanged, leads to a composition whose viscosity is lower than that of SEPIGEL® 305, and in particular lower than 100 cps. This composition is not a gel.

Sensory Analysis:

A sensory analysis of the compositions of Examples 1 to 6 (0.5% by weight of polymer) and of the composition of SEPIGEL® (1% by weight of polymer) at iso-viscosity is carried out with the participation of 25 panelists. The panelists evaluate the criteria listed in Table 4 below for each of these hydroalcoholic compositions, and assign them a score from 0 to 5 during the application and after the application of these compositions.

TABLE 4
Sensory analysis criteria

| | Criteria | 0 to 5 |
|---|---|---|
| During application | Clogging feeling | Not clogging to very clogging |
| | Smoothness | Not smooth to very smooth |
| | Freshness | Not fresh to very fresh |
| | Stringy appearance | Not stringy to very stringy |
| | Sticky feeling | Not sticky to very sticky |
| | Drying time | Very short to very long |
| After application | Greasy feeling | Not greasy to very greasy |
| | Sticky feeling | Not sticky to very sticky |
| | Softness | Not soft to very soft |

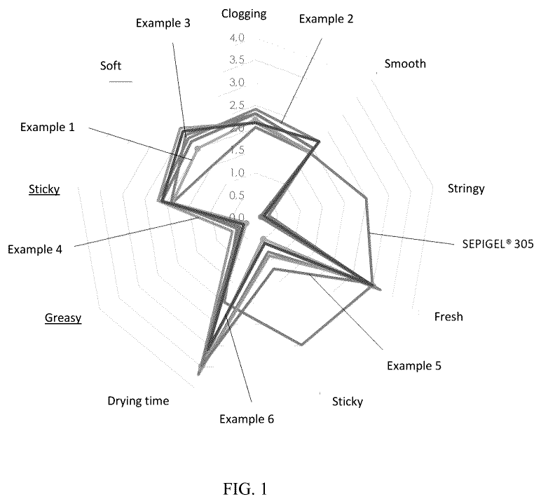

The results of the sensory analysis are illustrated in FIG. 1. In this FIGURE, the underlined criteria are evaluated after application of the composition, while the non-underlined criteria are evaluated during the application of the composition.

During the application, the polymers of Examples 1 to 6 of the invention and SEPIGEL® 305 have similar characteristics, namely the feeling of freshness is considered identical as well as the smoothness. On the other hand, the polymers of Examples 1 to 6 of the invention differ from SEPIGEL® 305 by a sticky feeling much less marked during the application of the composition, considered unpleasant, but above all by a total lack of stringy appearance during the application of the composition.

After applying the composition, the panelists judged that the compositions of Examples 1 to 6 of the invention left the skin softer but noted a longer drying time (on average 6s), the other parameters studied remaining identical.

The general impression tends towards better sensory qualities for the compositions prepared with Examples 1 to 6, compared to the composition prepared with SEPIGEL® 305.

1. Use, for the manufacture of a hydroalcoholic composition comprising 50% to 80% by weight of at least one alcohol and at least 10% by weight of an aqueous phase, of a branched or crosslinked polymer composed of the repetition of one or more monomeric units, wherein at least one of the monomeric units comprises an acrylic group, and at least 30 mol % of the monomeric units bear at least one weak acid function, possibly in neutralized form, said polymer being obtained:
by polymerization of an aqueous solution of one or more monomers in a water-in-oil inverse emulsion, wherein at least one of the monomers used is an acrylic monomer, and at least one of the monomers used is a monomer bearing at least one weak acid function, the molar percentage of monomers bearing at least one weak acid function relative to all the monomers used being at least 30%, the aqueous phase of the aqueous solution containing at least one monomer acting as a branching agent, so that the polymerization leads to a branched or crosslinked polymer, and wherein:
i) the polymerization is carried out with a concentration of all the monomers in aqueous solution belonging to the range from 1.3 mmol to 3.6 mmol per gram of aqueous solution,
ii) during the polymerization, at most 20 mol % of the acid functions present on the monomers having at least one acid function are in neutralized form.

2. The use of a branched polymer or crosslinked compound consisting of the repetition of one or more monomeric units to thicken a hydroalcoholic composition in order to obtain a thickened hydroalcoholic composition comprising 50% to 80% by weight of at least one alcohol and at least 10% by weight of an aqueous phase, wherein at least one of the monomeric units comprises an acrylic group and at least 30 mol % of the monomeric units bear at least one weak acid function, optionally in neutralized form, said polymer being obtained:
by polymerization of an aqueous solution of one or more monomers in a water-in-oil inverse emulsion, wherein at least one of the monomers used is an acrylic monomer and at least one of the monomers used is a monomer bearing at least one weak acid function, the molar percentage of monomers bearing at least one weak acid function relative to all the monomers used being at least 30%, the aqueous phase of the aqueous solution containing at least one monomer acting as a branching agent, so that the polymerization leads to a branched or crosslinked polymer, and wherein:
i) the polymerization is carried out with a concentration of all the monomers in aqueous solution belonging to the range from 1.3 mmol to 3.6 mmol per gram of aqueous solution,
ii) during the polymerization, at most 20 mol % of the acid functions present on the monomers having at least one acid function are in neutralized form.

3. The use according to claim 1, wherein the polymer comprises a percentage of neutralized acid functions of 30% to 100 mol % relative to all of the acid functions present on the polymer, obtained by an at least partial neutralization step of the acid functions present on the polymer, carried out after the polymerization, and before or after the preparation of the composition.

4. The use according to any of claim 1, wherein the hydroalcoholic composition comprises:
a) 50% to 80% by weight of said at least one alcohol,
b) 0.1% to 10% by weight of the branched or crosslinked polymer,
c) 10% to 49.9% by weight of water,
d) 0% to 10% by weight of at least one additive.

5. The use according to claim 1, wherein the hydroalcoholic composition comprises:
a) 60% to 75% by weight of at least one alcohol,
b) 0.1% to 5% by weight of the branched or crosslinked polymer,
c) 25% to 39.9% by weight of water,
d) 0% to 10% by weight of at least one additive.

6. The use according to claim 1, wherein the monomeric unit(s) bearing at least one weak acid function, in free form, is (are) chosen from acrylic acid, methacrylic acid, itaconic acid, crotonic acid, maleic acid and fumaric acid, acrylic acid being preferred.

7. The use according to claim 1, wherein the polymer is a copolymer comprising at least one neutral monomeric unit chosen from acrylamide, methacrylamide, N,N-dimethylacrylamide, N-vinylmethylacetamide, N-vinylformamide, vinyl acetate, diacetoneacrylamide, N-isopropyl acrylamide, N-[2-hydroxy-1,1-bis(hydroxymethyl)ethyl]propenamide, (2-hydroxyethyl) acrylate, (2,3-dihydroxypropyl) acrylate, methyl methacrylate, (2-hydroxyethyl) methacrylate, (2,3-dihydroxypropyl) methacrylate and vinylpyrrolidone.

8. The use according to claim 1, wherein the branching agent is selected from methylenebisacrylamide (MBA), ethylene glycol di-acrylate, polyethylene glycol dimethacrylate, diacrylamide, cyanomethylacrylate, vinyloxyethylacrylate, vinyloxymethacrylate, triallylamine, formaldehyde, glyoxal, glycidyl ethers such as ethylene glycol diglycidyl ether, epoxies and mixtures thereof.

9. The use according to claim 1, wherein the amount of branching agent is between 5 ppm and 10,000 ppm by weight relative to the total weight of the monomers, and preferably between 100 ppm and 5,000 ppm.

10. A hydroalcoholic composition, comprising 50% to 80% by weight of at least one alcohol, at least 10% by weight of an aqueous phase, and a branched or crosslinked polymer composed of the repetition of one or more monomeric units, wherein at least one of the monomer units comprises an acrylic group and at least 30 mol % of the monomer units bear at least one weak acid function, at least partially in neutralized form, said polymer being obtained:
by polymerization of an aqueous solution of one or more monomers in a water-in-oil inverse emulsion, wherein at least one of the monomers used is an acrylic monomer and at least one of the monomers used is a monomer bearing at least one weak acid function, the molar percentage of monomers bearing at least one weak acid function relative to all the monomers used being at least 30%, the aqueous phase of the aqueous solution containing at least one monomer acting as a branching agent, so that the polymerization leads to a branched or crosslinked polymer, and wherein:
i) the polymerization is carried out with a concentration of all the monomers in aqueous solution belonging to the range from 1.3 mmol to 3.6 mmol per gram of aqueous solution,
ii) during the polymerization, at most 20 mol % of the acid functions present on the monomers having at least one acid function are in neutralized form.

11. The hydroalcoholic composition according to claim 10, wherein the polymerization is followed by a step of at least partial neutralization of the acid functions present on the polymer, carried out before or after the incorporation of the polymer into the composition.

12. The hydroalcoholic composition according to claim 10, wherein the polymer comprises a percentage of neutralized acid functions of 30% to 100 mol % relative to all the acid functions present on the polymer, obtained by the neutralization step.

13. The hydroalcoholic composition according to claim 10, said composition comprising:
a) 50% to 80% by weight of said at least one alcohol,
b) 0.1% to 10% by weight of the branched or crosslinked polymer,
c) 10% to 49.9% by weight of water,
d) 0% to 10% by weight of at least one additive.

14. The hydroalcoholic composition according to claim 10, said composition comprising:
a) 60% to 75% by weight of at least one alcohol,
b) 0.1% to 5% by weight of the branched or crosslinked polymer,
c) 25% to 39.9% by weight of water,
d) 0% to 10% by weight of at least one additive.

15. The composition according to claim 10, wherein the monomeric unit(s) bearing at least one weak acid function, in free form, is (are) chosen from acrylic acid, methacrylic acid, itaconic acid, crotonic acid, maleic acid and fumaric acid, acrylic acid being preferred.

16. The composition according to claim 10, wherein the polymer is a copolymer comprising at least one neutral monomeric unit chosen from acrylamide, methacrylamide, N,N-dimethylacrylamide, N-vinylmethylacetamide, N-vinylformamide, vinyl acetate, diacetoneacrylamide, N-isopropyl acrylamide, N-[2-hydroxy-1,1-bis(hydroxymethyl)ethyl]propenamide, (2-hydroxyethyl) acrylate, (2,3-dihydroxypropyl) acrylate, methyl methacrylate, (2-hydroxyethyl) methacrylate, (2,3-dihydroxypropyl) methacrylate and vinylpyrrolidone.

17. The composition according to claim 10, wherein the branching agent is chosen from methylenebisacrylamide (MBA), ethylene glycol di-acrylate, polyethylene glycol dimethacrylate, diacrylamide, cyanomethylacrylate, vinyloxyethylacrylate, vinyloxymethacrylate, triallylamine, formaldehyde, glyoxal, glycidyl ethers such as ethylene glycol diglycidyl ether, epoxies and mixtures thereof.

18. The composition according to claim 10, wherein the amount of branching agent is between 5 ppm and 10,000 ppm by weight relative to the total weight of the monomers, and preferably between 100 ppm and 5,000 ppm.
